(19) United States
(12) Patent Application Publication
(10) Pub. No.: US 2006/0264411 A1
(43) Pub. Date: Nov. 23, 2006

(54) Control of biofilm formation

(76) Inventor: Gary R. Eldridge, Encinitas, CA (US)

Correspondence Address: THOMPSON COBURN, LLP, ONE US BANK PLAZA, SUITE 3500, ST LOUIS, MO, 63101, US

(21) Appl. No.: 11/133,858

(22) Filed: May 20, 2005

(51) Int. Cl. A61K 31/56; A61K 31/20

(52) U.S. Cl. 514/169; 514/559

(57) ABSTRACT

The present invention provides a method for reducing or preventing the invasion of a bacterium into a tissue comprising modulating the expression of a cysB gene in the bacterium. The present invention further provides an in vivo method for reducing or preventing the formation of a biofilm in a tissue comprising modulating expression of a cysB gene in a cell capable of biofilm formation. The present invention also provides a method for controlling or preventing a chronic bacterial infection in a subject in need thereof comprising modulating the expression of a cysB gene in a bacterium that causes the chronic bacterial infection.

### CROSS REFERENCE TO RELATED APPLICATIONS

This application claims priority from U.S. patent application Ser. No. 11/085,279, filed on Mar. 21, 2005, which claimed priority to U.S. provisional applications Ser. Nos. 60/587,680 (filed on Jul. 14, 2004) and 60/609,763 (filed on Sep. 14, 2004).

### FIELD OF THE INVENTION

The present invention generally relates to methods and compounds useful for reducing or preventing invasion of a bacterium into a tissue comprising modulating the expression of a cysB gene in the bacterium. The present invention also relates to an in vivo method for reducing or preventing the formation of a biofilm in a tissue and to a method for controlling or preventing a chronic bacterial infection.

### BACKGROUND

Chronic infections involving biofilms are serious medical problems throughout the world. For example, biofilms are involved in 65% of human bacterial infections. Biofilms are involved in prostatitis, biliary tract infections, urinary tract infections, cystitis, lung infections, sinus infections, ear infections, acne, rosacea, dental caries, periodontitis, nosocomial infections, open wounds, and chronic wounds.

Bacterial biofilms exist in natural, medical, and engineering environments. The biofilms offer a selective advantage to a microorganism to ensure its survival, or allow it a certain amount of time to exist in a dormant state until suitable growth conditions arise. Unfortunately, this selective advantage poses serious threats to animal health, especially human health.

Compounds that modify biofilm formation would have a substantial medical impact by treating many chronic infections, reducing catheter- and medical device-related infections, and treating lung and ear infections. The potential market for potent biofilm inhibitors is exemplified by the sheer number of cases in which biofilms contribute to medical problems. The inhibitors may also be used to cure, treat, or prevent a variety of conditions, such as, but are not limited to, arterial damage, gastritis, urinary tract infections, pyelonephritis, cystitis, otitis media, otitis extema, leprosy, tuberculosis, benign prostatic hyperplasia, chronic prostatitis, chronic lung infections of humans with cystic fibrosis, osteomyelitis, bloodstream infections, skin infections, open or chronic wound infections, cirrhosis, and any other acute or chronic infection that involves or possesses a biofilm.

In the United States, the market for antibiotics is greater than $8.5 billion. After cardiovascular therapeutics, the sales of antibiotics are the second largest drug market in the United States. The antibiotic market is fueled by the continued increase in resistance to conventional antibiotics. Approximately 70% of bacteria found in hospitals resist at least one of the most commonly prescribed antibiotics. Because biofilms appear to reduce or prevent the efficacy of antibiotics, introduction of biofilm inhibitors could significantly affect the antibiotic market.

Using the protection of biofilms, microbes can resist antibiotics at a concentration ranging from 1 to 1.5 thousand times higher than the amount used in conventional antibiotic therapy. During an infection, bacteria surrounded by biofilms are rarely resolved by the immune defense mechanisms of the host. Costerton, Stewart, and Greenberg, leaders in the field of biofilms, have proposed that in a chronic infection, a biofilm gives bacteria a selective advantage by reducing the penetration of an antibiotic into the depths of the tissue needed to completely eradicate the bacteria's existence.

Traditionally, antibiotics are discovered using the susceptibility test methods established by the National Committee for Clinical Laboratory Standards (NCCLS). The methods identify compounds that specifically affect growth or killing of bacteria. These methods involve inoculation of bacterial species into a suitable growth medium, followed by the addition of a test compound, and then plot of the bacterial growth over a time period post-incubation. These antibiotics would not be effective therapeutics against chronic infections involving biofilms because the NCCLS methods do not test compounds against bacteria in a preformed biofilm. Consistently, numerous publications have reported a difference in gene transcription in bacteria living in biofilms from bacteria in suspension, which further explains the failure of conventional antibiotics to eradicate biofilm infections (Sauer, K. et al. J. Bacteriol. 2001, 183:6579-6589).

Biofilm inhibitors can provide an alternative mechanism of action from conventional antibiotics. For example, successful treatment of nosocomial infections currently requires an administration of a combination of products, such as amoxicillin/clavulanate and quinupristin/dalfopristin, or an administration of two antibiotics simultaneously. In one study of urinary catheters, rifampin was unable to eradicate methicillin-resistant Staphylococcus aureus in a biofilm but was effective against planktonic, or suspended cells (Jones, S. M., et. al., “Effect of vancomycin and rifampicin on methicillin-resistant Staphylococcus aureus biofilms”, Lancet 357:40-41, 2001). Biofilm inhibitors act on the biological mechanisms that provide bacteria protection from antibiotics and from a host's immune system. Biofilm inhibitors may be used to “clear the way” for the antibiotics to penetrate the affected cells and eradicate the infection.

Moreover, bacteria have no known resistance to biofilm inhibitors. Biofilm inhibitors are not likely to trigger growth-resistance mechanisms or affect the growth of the normal human flora. Thus, biofilm inhibitors could potentially extend the product life of antibiotics.

Biofilm inhibitors can also be employed for the treatment of acne. Acne vulgaris is the most common cutaneous disorder. Propionibacterium acnes, which is the predominant microorganism occurring in acne, reside in biofilms. Its existence in a biofilm matrix provides a protective, physical barrier that limits the effectiveness of antimicrobial agents (Burkhart, C. N. et. al., “Microbiology's principle of biofilms as a major factor in the pathogenesis of acne vulgaris”, International J. of Dermatology. 42:925-927, 2003). Biofilm inhibitors may be used to effectively prevent, control, reduce, or eradicate P. acnes biofilms in acne.

Plaque biofilms contribute to cavities and and periodontitis. Plaque biofilms accumulate due to bacterial colonization of Streptococci spp. such as S. mutans, S. sobrinas, S. gordonii, and Porphyromonas gingivalis, and Actinomyces spp (Demuth, D. et al. Discrete Protein Determinant Directs the Species-Species Adherence of Porphyromonas gingivalis to Oral Streptococci, Infection and Immunity, 2001, 69(9) p 5736-5741; Xie, H., et al. Intergeneric Communication in Dental Plaque Biofilms. J. Bacteriol. 2000, 182(24), p 7067-7069). The primary colonizing bacteria of plaque accumulation are Streptococci spp., and P. gingivalis is a leading cause of periodontitis (Demuth, D. et al. Discrete Protein Determinant Directs the Species-Species Adherence of Porphyromonas gingivalis to Oral Streptococci, Infection and Immunity, 2001, 69(9) p 5736-5741). Biofilm inhibitors can be employed to prevent microorganisms from adhering to surfaces that may be porous, soft, hard, semi-soft, semi-hard, regenerating, or non-regenerating. These surfaces can be teeth, the polyurethane material of central venous catheters, or metal, alloy, or polymeric surfaces of medical devices, or regenerating proteins of cellular membranes of mammals, or the enamel of teeth. These inhibitors can be coated on or impregnated into these surfaces prior to use, or administered at a concentration surrounding these surfaces to control, reduce, or eradicate the microorganisms adhering to these surfaces.

Chronic wound infections are difficult to eradicate or routinely recur. Diabetic foot ulcers, venous leg ulcers, arterial leg ulcers, and pressure ulcers are examples of the most common types of chronic wounds. Diabetic foot ulcers appear to be the most prevalent. These wounds are typically colonized by multiple species of bacteria including Staphylococcus spp., Streptococcus spp., Pseudomonas spp. and Gram-negative bacilli (Lipsky, B. Medical Treatment of Diabetic Foot Infections. Clin. Infect. Dis. 2004, 39, p. S104-14). Based on clinical evidence, researchers know that multiple microorganisms can cause or contribute to chronic wound infections. Only recently have biofilms been implicated in these infections (Harrison-Balestra, C. et al. A Wound-isolated Pseudomonas aeruginosa Grow a Biofilm In Vitro Within 10 Hours and Is Visualized by Light Microscopy. Dermatol Surg 2003, 29, p. 631-635; Edwards, R., et al. Bacteria and wound healing. Curr Opin Infect Dis, 2004, 17, p. 91-96). In fact, it is estimated that approximately 140,000 amputations occur each year in the United States due to chronic wound infections that could not be treated with conventional antibiotics. Unfortunately, treating these infections with high doses of antibiotics over long periods of time can contribute to the development of antibiotic resistance (Howell-Jones, R. S., et al. A review of the microbiology, antibiotic usage and resistance in chronic skin wounds. J. Antimicrob. Ther. January 2005). Biofilm inhibitors in a combination therapy with antibiotics may provide an alternative to the treatment of chronic wounds.

Recent publications describe the cycles of the pathogenesis of numerous species of bacteria involving biofilms. For example, Escherichia coli, which causes recurrent urinary tract infections, undergo a cycle of binding to and then invading bladder epithelial cells, forming a biofilm intracellularly, modifying its morphology intracellularly, and then bursting out of cells to repeat the cycle of pathogenesis (Justice, S. et al. Differentiation and development pathways of uropathogenic Escherichia coli in urinary tract pathogenesis. PNAS, 2004, 101(5): 1333-1338). The authors suggest that this repetitive cycle of pathogenesis of E. coli may explain the recurrence of the infection.

In 1997 Finlay, B. et al. reported that numerous bacteria, including Staphylococci, Streptococci, Bordetella pertussis., Neisseria spp., Helicobactor pylori, Yersinia spp. adhere to mammalian cells during their pathogenesis. The authors hypothesized that the adherence would lead to an invasion of the host cell. Later publications confirm this hypothesis (Cossart, P. Science, 2004, 304, p. 242-248; see additional references below). A few of these publications presented hypotheses similar to Mulvey, M, et al, which explained the invasion of these bacteria into cells. (Mulvey, M, et al. “Induction and Evasion of Host Defenses by Type 1-Piliated Uropathogenic E. coli” Science 1998, 282 p. 1494-1497). Mulvey, M. et al. stated invasion of E. coli into epithelial cells provide protection from the host's immune response to allow a build up of a large bacterial population.

Cellular invasion and biofilms appear to be integral to the pathogenesis of most, if not all bacteria. Pseudomonas aeruginosa has been shown to invade epithelial cells during lung infections (Leroy-Dudal, J. et al. Microbes and Infection, 2004, 6, p. 875-881). P. aeruginosa is the principal infectious organism found in the lungs of cystic fibrosis patients, and the bacteria exist within a biofilm. Antibiotics like tobramcyin, and current antibacterial compounds do not provide effective treatment against biofilms of chronic infections, because antibiotic therapy fails to eradicate the biofilm.

Gram-negative bacteria share conserved mechanisms of bacterial pathogenesis involving cellular invasion and biofilms. For example, Haemophilus influenzae invade epithelial cells and form biofilms (Hardy, G. et al., Methods Mol. Med., 2003, 71, p. 1-18; Greiner, L. et al., Infection and Immunity, 2004, 72(7) p. 4249-4260). Burkholderia spp. invade epithelial cells and form biofilm (Utaisincharoen, P, et al. Microb Pathog. 2005, 38(2-3) p. 107-112; Schwab, U. et al. Infection and Immunity, 2003, 71(11), p. 6607-6609). Klebsiella pneumoniae invade epithelial cells and form biofilm (Cortes, G et al. Infection and Immunity. 2002, 70(3), p. 1075-1080; Lavender, H, et al. Infection and Immunity. 2004, 72(8), p. 4888-4890). Salmonella spp. invade epithelial cells and form biofilms (Cossart, P. Science, 2004, 304, p. 242-248; Boddicker, J. et al. Mol. Microbiol. 2002, 45(5), p. 1255-1265). Yersinia pestis invade epithelial cells and form biofilms (Cossart, P. Science, 2004, 304, p. 242-248; Jarrett, C. et al. J. Infect. Dis., 2004, 190, p. 783-792). Neisseria gonorrhea invade epithelial cells and form biofilms (Edwards, J. et al., Cellular Micro., 2002, 4(9), p. 585-598; Greiner, L. et al. Infection and Immunity. 2004, 73(4), p. 1964-1970).

These Gram-negative bacteria cause lung, ear, and sinus infections, gonorrhoeae, plague, diarrhea, typhoid fever, and other infectious diseases. E. coli and P. aeruginosa are two of the most widely studied Gram-negative pathogens. Researchers believe that the pathogenesis of these bacteria involves invasion of host cells and formation of biofilms. These models have enabled those skilled in the art to understand the pathogenesis of other Gram-negative bacteria.

Gram-positive bacteria also share conserved mechanisms of bacterial pathogenesis involving cellular invasion and biofilms. Staphylococcus aureus invade epithelial cells and form biofilms (Menzies, B, et al. Curr Opin Infect Dis, 2003, 16, p. 225-229; Ando, E, et al. Acta Med Okayama, 2004, 58(4), p. 207-14). Streptococcus pyogenes invade epithelial cells and form biofilms (Cywes, C. et al., Nature, 2001,414, p. 648-652; Conley, J, et al. J. Clin. Micro., 2003, 41(9), p. 4043-4048).

Accordingly, for the reasons discussed above and others, there exists an unmet need for methods and compounds that can reduce or prevent the invasion of bacteria and the formation of biofilm in human cells.

### SUMMARY OF INVENTION

Accordingly, the present invention provides a method for reducing or preventing the invasion of a bacterium into a tissue comprising modulating the expression of a cysB gene in the bacterium.

The present invention further provides an in vivo method for reducing or preventing the formation of a biofilm in a tissue comprising modulating expression of a cysB gene in a cell capable of biofilm formation.

The present invention also provides a method for controlling or preventing a chronic bacterial infection in a subject in need thereof comprising modulating the expression of a cysB gene in a bacterium that causes or contributes to the chronic bacterial infection.

### BRIEF DESCRIPTION OF THE DRAWINGS

FIG. 1 shows the chemical synthesis of an analog of ursolic acid.

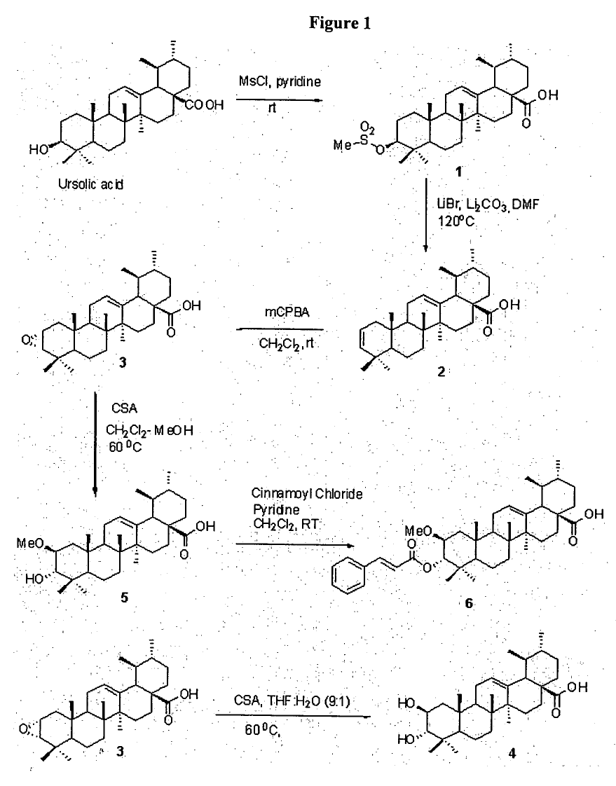

FIG. 2 shows a confocal microscopy image of an IBC of E coli from a bladder of a control mouse inoculated with E. coli UTI89.

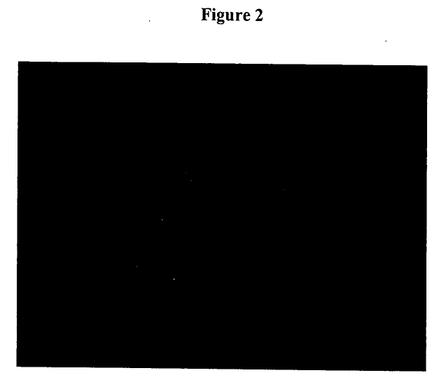

FIG. 3 shows a confocal microscopy image of a small collection of E coli from a bladder of a mouse inoculated with E. coli UTI89 and corosolic acid.

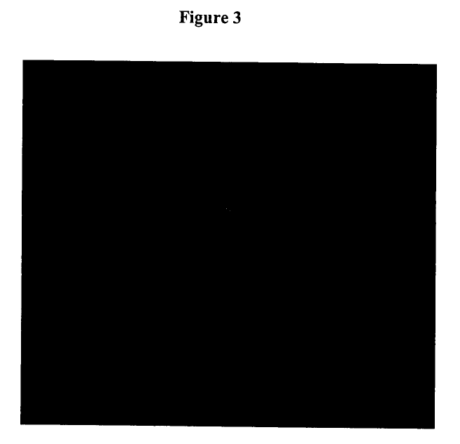

FIG. 4 shows a confocal microscopy image of an IBC from a bladder of a mouse inoculated with wild type E. coli UTI89.

FIG. 5 shows a confocal microscopy image of a loose collection of E coli from a bladder of a mouse inoculated with 50:50 E. coli UTI89 cysB⁻/wild type E. coli UIT89.

FIG. 6 shows a confocal microscopy image of a loose collection of E coli from a bladder of a mouse inoculated with 50:50 E. coli UTI89 cysB⁻/wild type E. coli UIT89.

### DESCRIPTION OF THE INVENTION

### Definitions

“Acceptable carrier” refers to a carrier that is compatible with the other ingredients of the formulation and is not deleterious to the recipient thereof.

“Reducing or inhibiting” in reference to a biofilm refers to the prevention of biofilm formation or growth, reduction in the rate of biofilm formation or growth, partial or complete inhibition of biofilm formation or growth.

“Modulates” or “modulating” refers to up-regulation or down-regulation of a gene's replication or expression.

The present invention provides a method for reducing or preventing the invasion of a bacterium into a tissue comprising modulating the expression of a cysB gene in the bacterium.

The cysB gene may be modulated in a number of ways. For example, N-acetyl-serine and sulfur limitation up-regulate cysB. Lochowska, A. et al., Functional Dissection of the LysR-type CysB Transcriptional Regulator. J. Biol. Chem. 2001, 276, 2098-2107. In addition, like other LysR type regulators, cysB can repress itself. Lilic, M. et al., Identification of the CysB-regulated gene, hslJ, related to the Escherichia coli novobiocin resistance phenotype, FEMS Micro. Letters. 2003, 224:239-246.

In one embodiment, a tissue is contacted with a composition comprising a compound selected from the group consisting of ursolic acid or asiatic acid, or a pharmaceutically acceptable salt of such compound, or hydrate of such compound, or solvate of such compound, an N-oxide of such compound, or combination thereof. In a preferred embodiment, the compound is corosolic acid, 30-hydroxyursolic acid, 20-hydroxyursolic acid, 2-hydroxyoleanolic acid, and madecassic acid. In another preferred embodiment, the compound is pygenic acid (A, B, or C), euscaphic acid, and tormentic acid.

The compounds used in the present invention may be isolated from a plant as previously described or prepared semi-synthetically (Eldridge, G, et al; Anal. Chem. 2002, 74, p. 3963-3971). If prepared semi-synthetically, a typical starting material may be ursolic acid, oleanolic acid, corosolic acid, asiatic acid, madecassic acid or other compound used in the present invention. In designing semi-synthetic strategies to prepare analogs, certain positions of the scaffold of the compounds are important for modulating biofilm inhibition, while other positions improve bioavailability of the compounds, which could expand the therapeutic range of the compounds by reducing certain cellular toxicities in mammals.

Herbal preparations of Centella asiatica plant extracts, which contain hundreds to thousands of compounds, have been used throughout history in numerous countries for the treatment of dermatological conditions, including wound healing, such as burns and scar reduction. Herbal preparations of Centella asiatica plant extracts have also be used to treat asthma, cholera, measles, diarrhea, epilepsy, jaundice, syphilis, and cystitis. These herbal preparations are commercially available. The preparations may include asiaticoside, madecassoside, brahmoside, brahminoside, asiatic acid, and madecassic acid. Syntex Research Centre, for example, marketed a titrated plant extract of Centella asiatica for the treatment of burns; the extract contained asiatic acid, madecassic acid, and asiaticoside. However, the commercially-available herbal preparations of Centella asiatica plant extracts are not pure compounds. Those skilled in the art have not been able to determine which pure compounds in the extracts are responsible for the medicinal benefits.

As previously demonstrated in the examples of U.S. patent application Ser. No. 11/085,279, ursolic acid and asiatic acid modulate the expression of a cysB gene in E. coli. In modulating the cysB gene, the compound could also modulate the expression of genes under the control or within the same biochemical pathway as cysB. The cysB protein is a transcriptional regulator of the LysR family of genes. The transcriptional regulators of this family have helix-turn-helix DNA binding motifs at their amino-terminus. The cysB protein is required for the full expression of the cys genes, which are involved in the biosynthesis of cysteine. The family of genes, cysDIJK are under the transcriptional control of the cysB gene. cysD, cysI, cysJ, and cysK are proteins involved in the biosynthesis of cysteine. CysK has been shown to respond to extracellular signals in bacteria (Sturgill, et al. J. Bacteriol. 2004 ,186(22) p. 7610-7617). YbiK is under the direct control of cysB and participates in glutathione intracellular transport. b0829 is involved in glutathione transport. b1729 is suspected to be a carboxylate transporter based upon sequence homology. b1729 is conserved amongst Gram-negative and Gram-positive bacteria (http://wvvw.ncbi.nlm.nih.gov/sutils/genomtable.cgi). Accordingly, preferably, the compound used in the present invention modulates the expression of cysD, cysI, cysJ, cysK, ybiK, b0829, b1729, yeeD, and/or yeeE.

Members of the family of LysR transcriptional regulators, like CysB, have been demonstrated to regulate diverse metabolic processes. cysB exhibits direct control of the biosynthesis of cysteine (Verschueren et al., at p. 260). The cysB gene is involved, directly or indirectly, in glutathione intracellular transport, carbon source utilization, alanine dehydrogenases, and the arginine dependent system. There is also recently published evidence that suggests that cysB responds directly or indirectly to extracellular signals (Sturgill, et al. J. Bacteriol. 2004, 186(22) p. 7610-7617). CysB regulates the expression of CysK, cysM, cysA, which are closely linked to crr, ptsI, and ptsH (Byrne, et al. J. Bacteriol. 170(7) p. 3150-3157). PtsI has been implicated in the sensing of external carbohydrates (Alder, et al. PNAS, 1974, 71, p. 2895-2899).

In one embodiment, the cysB gene in a Gram-negative bacterium is modulated. Preferably, the bacterium is Escherichia coli, Pseudomonas aeruginosa, Haemophilus influenzae.

As previously discussed herein, Gram-positive and Gram-negative bacteria invade their cellular hosts through conserved mechanisms of bacterial pathogenesis. The process enables the bacteria to evade the hosts' immune responses to allow the bacteria to increase their population. Therefore, compounds which can reduce bacterial invasion would significantly assist the immune system in the eradication of these pathogens. A reduction in bacterial invasion into cells would also increase the efficiency and potency of conventional antibiotics. Niels Moller-Frimodt demonstrated that antibiotics efficiently killed bacteria in the urine in a urinary tract infection, but were less effective in killing the bacteria in the bladder or tissues (Moller-Frimodt, N. Int. J. of Antimicrob Agents, 2002, 19, p. 546-553).

The cysB gene is genetically conserved among different species of bacteria, such as Gram-negative bacteria. Verschueren, et al., Acta Cryst. (2001) D57, 260-262; Byrne et al., J. Bacteriol. 1988 170(7):3150-3157. In fact, cysB is conserved among Pseudomonas sp. including, but not limited to, P. aeruginosa, P. putida, and P. syringae. (http://www.ncbi.nlm.nih.gov/sutils/genom_table.cgi). (Blast search of the cysB gene at the Microbial Genomics database at the National Center for Biotechnology Information (NCBI) of the National Institutes of Health (NIH)). The cysB gene is also genetically conserved among the following species of bacteria: Vibrio sp. (e.g. V. harveyi and V. cholera), Proteus mirablis, Burkholderia sp. (e.g. B. fongorum, B. mallei, and B. cepacia), Klebsiella sp., Haemophilus influenza, Neisseria meningitides, Bordetella pertussis, Yersinia pestis, Salmonella typhimurium, and Acinetobacter sp. (http://www.ncbi.nlm.nih.gov/sutils/genom_table.cgi. Blast search of the cysB gene at the Microbial Genomics database at NCBI of NIH). The cysB gene is also genetically conserved among the Gram-positive bacteria of Bacillus sp. including, but not limited to, B. subtilis, B. cereus, and B. anthracis. (http://www.ncbi.nlm.nih.gov/sutils/genom table.cgi). (Blast search of the cysB gene at the Microbial Genomics database at NCBI of NIH; van der Ploeg, J. R.; FEMS Microbiol. Lett. 2001, 201:29-35).

The cysB gene is involved in the invasion of a bacterium into a cell. The cell may be mammalian cells, preferably epithelial cells. As demonstrated in the examples herein, the removal of a cysB gene from E. coli resulted in a significant reduction in invasion of E. coli into bladder epithelial cells as compared to wild-type E. coli.

In another embodiment, the method reduces or prevents the invasion of a bacterium into a mammalian tissue. Preferably, the mammalian tissue is a murine tissue. More preferably, the mammalian tissue is a human tissue. Still preferably, the human tissue is a bladder, a kidney, or a prostate.

It has been previously shown that E. coli invades the kidney and prostate of humans. E. coli causes pyelonephritis and prostatitis, which are infectious diseases that can lead to death. (Russo, T., et al. Medical and economic impact of extraintestinal infections due to Escherichia coli: focus on an increasingly important endemic problem. Microbes and Infection. 2003, 5, p. 449-456). Research shows that E. coli uses the same or similar mechanism to invade kidney and prostate in humans to cause these infections as it does to cause urinary tract infections. Therefore, a person of ordinary skill in the art would reasonably conclude that modulating a cysB gene in E. coli with the compounds described in the specification could also prevent invasion and reduce the formation of biofilms in kidneys and prostate.

In still another embodiment, the method reduces or prevents the invasion of a bacterium into a plant tissue. Gram-negative bacteria invade and colonize plants. The compounds of the invention that modulate cysB can be isolated from a very few plants, but to date it has not been shown that they can be isolated from commercial food crops or ornamental plants. Pseudomonas putida, a Gram-negative bacterium, forms biofilms on plants (Arevalo-Ferro, C; Biofilm formation of Pseudomonas putida IsoF: the role of quorum sensing as assessed by proteomics. Syst. Appl. Microbiol. 2005, 28(2) p. 87-114.) Plants that produce the compounds used in the present invention have probably evolved to make these compounds to reduce, prevent, or control the invasion of bacteria and the formation of biofilms.

The present invention further provides an in vivo method for reducing or preventing the formation of a biofilm in a tissue comprising modulating expression of a cysB gene in a cell capable of biofilm formation.

As demonstrated by the examples herein, cysB plays a significant role in the formation of biofilms and the invasion of bacteria into mammalian cells. Therefore, the cysB gene is vital for the pathogenesis of bacteria. Compounds used in the present invention reduce the formation of biofilms and reduce or prevent the invasion of bacteria into mammalian cells. The compounds modulate the expression of a cysB gene in a cell capable of biofilm formation.

In an embodiment, the in vivo method comprises contacting the tissue with a composition comprising a compound selected from the group of ursolic acid, or asiatic acid, or a pharmaceutically acceptable salt of such compound, or hydrate of such compound, or solvate of such compound, an N-oxide of such compound, or combination thereof.

Example 5 show that asiatic acid, corosolic acid and madecassic acid, along with an antibiotic, can reduce the sustainability of pre-formed biofilms. Because biofilm contributes to many chronic bacterial infections, these examples strongly support the use of the compounds of the present invention to treat chronic bacterial infections, such as lung and ear infections and diabetic foot ulcers. The results of the examples demonstrate the distinct difference between the methods used to discover biofilm inhibitors and the NCCLS methods used to discover conventional antibiotics. Not surprisingly, the NCCLS method fails to identify antibiotics that can effectively treat chronic infections involving biofilms.

In a preferred embodiment, the compound is corosolic acid, 30-hydroxyursolic acid, 20-hydroxyursolic acid, 2-hydroxyoleanolic acid, and madecassic acid. In another preferred embodiment, the compound is pygenic acid (A, B, or C), euscaphic acid, and tormentic acid.

By modulating the cysB gene, the compound could also modulate the expression of genes under the control or within the same biochemical pathway as cysB. Preferably, the compound modulates the expression of cysD, cysI, cysJ, cysK, ybiK, b0829, b1729, yeeD, and/or yeeE.

In one embodiment of the present invention, the cysB gene in a Gram-negative bacterium is modulated. Preferably, the bacterium is Escherichia coli, Pseudomonas aeruginosa, Haemophilus influenzae.

Examples 1, 4, 5, and 6 demonstrate that the compounds of the present invention serve as biofilm inhibitors by reducing the attachment of Pseudomonas aeruginosa, Escherichia coli, Streptococcus mutans, and Streptococcus sobrinas to surfaces. The compounds prevent, reduce or inhibit biofilm across a broad spectrum of bacteria. The present invention demonstrates that asiatic acid, corosolic acid, madecassic acid exhibit inhibition or reduction of biofilm of bacteria that are genetically diverse from each other. These bacteria may be Gram-positive or Gram-negative and may beclinical or laboratory strains. The examples also specifically demonstrate that asiatic acid, corosolic acid and madecassic acid can reduce a mature biofilm with antibiotic.

In another embodiment, the method reduces or prevents formation of a biofilm in a mammalian tissue. Preferably, the mammalian tissue is a murine tissue. More preferably, the mammalian tissue is a human tissue. Still preferably, the human tissue is a bladder, a kidney, or a prostate.

In still another embodiment, the method reduces or prevents formation of a biofilm in a plant tissue.

The present invention also provides a method for controlling or preventing a chronic bacterial infection in a subject in need thereof comprising modulating the expression of a cysB gene in a bacterium that causes or contributes to the chronic bacterial infection.

Biofilm inhibitors will have a substantial medical impact by treating many chronic infections, reducing catheter- and medical device-related infections, and treating lung and ear infections. Biofilm inhibitors may be used to control microorganisms existing extracellularly or intracellularly of living tissues. They may be used to cure, treat, or prevent a variety of conditions, such as, but are not limited to, arterial damage, gastritis, urinary tract infections, otitis media, leprosy, tuberculosis, benign prostatic hyperplasia, cystitis, pyeolonephritis, prostatitis, lung, ear, and sinus infections, periodontitis, cirrhosis, osteomyelitis, bloodstream infections, skin infections, acne, rosacea, open or chronic wound infections, and any other acute or chronic infection that involves or possesses a biofilm.

In an embodiment of the present invention, the modulation of the cysB gene comprises administering to a subject in need thereof with an effective amount of a composition comprising a compound selected from the group consisting of ursolic acid or asiatic acid, or a pharmaceutically acceptable salt of such compound, or hydrate of such compound, or solvate of such compound, an N-oxide of such compound, or combination thereof.

In a preferred embodiment, the compound is corosolic acid, 30-hydroxyursolic acid, 20-hydroxyursolic acid, 2-hydroxyoleanolic acid, and madecassic acid. In another preferred embodiment, the compound is pygenic acid (A, B, or C), euscaphic acid, and tormentic acid.

By modulating the cysB gene, the compound could also modulate the expression of genes under the control or within the same biochemical pathway as cysB. Preferably, the compound modulates the expression of cysD, cysI, cysJ, cysK, ybiK, b0829, b1729, yeeD, and/or yeeE.

In an embodiment of the present invention, the chronic bacterial infection is selected from the group consisting of urinary tract infection, gastritis, lung infection, ear infection, cystitis, pyelonephritis, arterial damage, leprosy, tuberculosis, benign prostatic hyperplasia, prostatitis, osteomyelitis, bloodstream infection, cirrhosis, skin infection, acne, rosacea, open wound infection, chronic wound infection, and sinus infection.

Example 7 demonstrates how the compounds of the present invention interrupt, delay, or prevent the cycle of pathogenesis of other E. coli infections such as, but not limited to, pyelonephritis, prostatitis, meningitis, sepsis, and gastrointestinal infections.

In another embodiment of the present invention, the chronic bacterial infection results from an infection of a bacterium. Preferably, the bacterium is a Gram-negative bacterium. More preferably, the bacterium is Escherichia coli, Pseudomonas aeruginosa, or Haemophilus influenzae.

In still another embodiment of the present invention, the chronic bacterial infection causes an autoimmune disease in a mammal. Preferably, the mammal is a human.

Recent scientific research demonstrates that certain diseases may be caused by bacteria that cannot be detected using current technology. For example, U.S. patent application no. 20050042214 describes new strains of bacteria that are ubiquitous and that metabolize complex organic chemical compounds. In particular, Novosphingobium aromaticivorans, a Gram-negative bacteria, was discovered and classified within the Sphingomonas genus. The bacteria appeared to be involved in primary biliary cirrhosis, an autoimmune disease. The bacteria may also play a critical role in other autoimmune diseases such as CRST syndrome (calcinosis, Raynaud's phenomenon, sclerodactyly, telangiectasia), the sicca syndrome, autoimmune thyroiditis, or renal tubular acidosis, ankylosing spondylitis, antiphospholipid syndrome, Crohn's disease, ulcerative colitis, insulin dependent diabetes, fibromyalgia, Goodpasture syndrome, Grave's disease, lupus, multiple sclerosis, myasthenia gravis, myositis, pemphigus vulgaris, rheumatoid arthritis, sarcoidosis, scleroderma, or Wegener's granulomatosis. Similar to other Gram-negative bacteria, the pathogenesis of N. aromaticivorans most likely involves the modulation of a cysB gene. Therefore, it is reasonable to conclude that the present invention may be used to treat autoimmune diseases caused by bacteria that invade and live within a protective biofilm.

Veeh et al. recently demonstrated that conventional microbiology techniques failed to detect colonization of bacteria on some human tissues (Veeh, et al. J. Infect. Dis. 2003, 188, p. 519-530). With new molecular biology techniques, such as PCR and FISH (fluorescent in situ hybridization), more bacteria living in biofilms are discovered. For example, new techniques show the prevalence of vaginal Staphylococcus aureus living in biofilms. As technology advances, researchers may uncover additional bacteria living in biofilms that cause or contribute to diseases. The present invention may also be used to treat these diseases.

### EXAMPLES

The following examples illustrate the testing of compounds of the present invention and the preparation of formulations comprising these compounds. The examples demonstrate the many uses of the compounds and are not intended to limit the scope of the present invention.

### Example 1

Biofilm Formation of Asiatic acid, Corosolic acid, and Madecassic Acid against Escherichia coli Clinical Strain UTI89 and Laboratory Strain JM109.

Biofilm inhibition experiments were conducted using an assay adapted from the reported protocol described in Pratt and Kolter, 1998, Molecular Microbiology, 30: 285-293; Li et al., 2001, J. Bacteriol., 183: 897-908. E. coli clinical strain UTI89 was grown in LB in 96 well plates at room temperature for one or two days without shaking. E. coli laboratory strain JM109 was grown in LB plus 0.2% glucose in 96 well plates at room temperature for one day without shaking. To quantify the biofilm mass, the suspension culture was poured out and the biofilm was washed three times with water. The biofilm was stained with 0.1% crystal violet for 20 minutes. The plates were then washed three times with water. OD reading at 540 nm was measured to quantify the biofilm mass at the bottom of the wells. Then 95% ethanol was added to dissolve the dye at the bottom and on the wall and the OD reading at 540 nm was measured to quantify the total biofilm mass. To study the overall effect of the compounds (3.6 mg/mL in 100% ethanol as stock solution), it was added with the inoculation and a time course of biofilm mass was measured. Appropriate amounts of 100% ethanol were added to each sample to eliminate the effect of solvent. Each condition had 3-4 replicates on each plate and was performed over multiple days.

The compounds tested had no inhibitory effect on the growth of either strain of E. coli when compared to controls, demonstrating that these compounds are not antibacterial compounds. Asiatic acid inhibited biofilm formation of the UTI89 strain by about 90%, 50%, 15%, and 10% as compared to the controls at 32, 16, 8, and 4 ug/ml, respectively. Corosolic acid inhibited biofilm formation of the UTI89 strain by about 85% at 20 ug/ml. Asiatic acid inhibited biofilm formation of the JM109 strain by about 80% and 70% as compared to the controls at 10 and 5 ug/ml, respectively. Madecassic acid inhibited biofilm formation of the JM109 strain by about 75% and 60% as compared to the controls at 10 and 5 ug/ml, respectively. These experiments confirm that asiatic acid, corosolic acid, madecassic acid, and the compounds of the invention inhibit the formation of biofilms against clinical and laboratory strains of E. coli.

### Example 2

Biofilm Formation in a cysB Deletion Mutant of E. coli Clinical Strain UTI89

An isogenic cysB deletion mutant was prepared from E. coli clinical strain UTI89. Briefly, the construction of a cysB deletion strain was prepared as follows: the red-recombinase method was utilized (Murphy, K. C., and K. G. Campellone. 2003. Lambda Red-mediated recombinogenic engineering of enterohemorrhagic and enteropathogenic E. coli. BMC Mol Biol 4:11). Using the template pKD4, a linear knockout product was generated using PCR and the primers 5′-ACGATGTTCTGATGGCGTCTAAGTGGATGGTTTAACATGAAATTACAACAAC TTCGGTGTAGGCTGGAGCTGCTTC-3′ and 5′-TCCGGCACCTTCGCTACATAAA AGGTG CCGAAAATAACGCAAGAAATTATTTTTCATGGGAATTAGC CATGGTCC-3′. The product was electroporated into red-recombinase expressing UTI89. The resultant strain had a complete deletion of the cysB coding sequence replaced by a kanamycin cassette. The resistance marker was secondarily excised from the chromosome by transformation with pCP20 expressing the FLP recombinase (Datsenko, K. A., and B. L. Wanner. 2000. One-step inactivation of chromosomal genes in Escherichia coli K-12 using PCR products. Proc Natl Acad Sci USA 97:6640-5). The appropriate chromosomal deletion were confirmed using cysB ORF flanking primers

5′-GAGTGTAAAAACACACGTA AGATTTTACGTAACGG-3′
and
5′-AAAACCGCCAGCCAGGCTTTACGTTT-3′.

Using the method described in Example 1, the formation of biofilms comparing this mutant strain, E. coli UTI89 cysB⁻, to wild type E. coli clinical strain UTI89 was examined. The growth of E. coli UTI89 cysB⁻ and wild type E. coli clinical strain UTI89 in LB medium were similar as determined by OD.

The mutant cysB strain of E. coli made 75% (n=8) and 66% (n=8) less biofilm as compared to wild type E. coli when tested on separate days. These experiments confirmed cysB's role in biofilm formation in clinical strains of E. coli, which are independent of bacterial growth in LB medium.

### Example 3

Antibacterial Effect of Asiatic Acid on Haemophilus influenzae (ATCC 10211), E. coli (ATCC 25922), and P. aeruginosa (ATCC 27853).

Using the appropriate NCCLS procedures, the antibacterial effect of asiatic acid on Haemophilus influenzae (ATCC 10211), E. coli (ATCC 25922), and P. aeruginosa (ATCC 27853) was studied at 64 μg/mL. Asiatic acid had no inhibitory effect represented by a MIC (minimal inhibitory concentration) of greater than 64 μg/ml. These results along with the results described in Example 2, further supports that asiatic acid is not an antibacterial compound.

### Example 4

Effect of Asiatic Acid on Mature Biofilms of Clinical Isolates of P. aeruginosa

Clinical isolates of P. aeruginosa from cystic fibrosis patients were passed twice on tryptic soy agar with 5% sheep blood after retrieval from −80° C. and then grown overnight in CAMHB. After dilution of a culture to 0.5 McFarland in broth medium, 100 μl was transferred in triplicate to wells of a flat-bottom 96-well microtiter plate. Bacterial biofilms were formed by immersing the pegs of a modified polystyrene microtiter lid into this biofilm growth plate, followed by incubation at 37° C. for 20 hours with no movement.

Peg lids were rinsed three times in sterile water, placed onto flat-bottom microtiter plates containing biofilm inhibitors at 5 ug/ml in 100 μl of CAMHB per well and incubated for approximately 40 hours at 37° C.

Pegs were rinsed, placed in a 0.1% (wt/vol) crystal violet solution for 15 min, rinsed again, and dried for several hours. To solubilize adsorbed crystal violet, pegs were incubated in 95% ethanol (150 μl per well of a flat-bottom microtiter plate) for 15 min. The absorbance was read at 590 nm on a plate reader. The wells containing asiatic acid were compared to negative controls. Negative controls were prepared as stated above but without asiatic acid.

Asiatic acid caused an average detachment of mature biofilms of approximately 50% at 5 ug/ml compared to the negative controls against eighteen clinical isolates of P. aeruginosa. The range of detachment of mature biofilms against all eighteen clinical isolates was 25% to 74%. This example demonstrates the ability of asiatic acid and the compounds of the invention to reduce mature biofilms in clinical isolates of P. aeruginosa.

### Example 5

Effect of Asiatic acid, Corosolic acid, or Madecassic Acid in Combination with Tobramycin on Biofilm Formation of Pseudomonas aeruginosa.

Biofilm formation of P. aeruginosa was evaluated using a standardized biofilm method with a rotating disk reactor (RDR). This method provides a model resembling the formation of biofilms in cystic fibrosis patients. The rotating disk reactor consists of a one-liter glass beaker fitted with a drain spout. The bottom of the vessel contains a magnetically driven rotor with six 1.27 cm diameter coupons constructed from polystyrene. The rotor consists of a star-head magnetic stir bar upon which a disk was affixed to hold the coupons. The vessel with the stir bar was placed on a stir plate and rotated to provide fluid shear. A nutrient solution (AB Trace Medium with 0.3 mM glucose, see Table 1 below for composition) was added through a stopper in the top of the reactor at a flow rate of 5 ml/min. The reactor volume was approximately 180 ml and varied slightly between reactors depending on the placement of the drain spout and the rotational speed of the rotor. At a volume of 180 ml, the residence time of the reactors was 36 minutes. The reactors were operated at room temperature (c.a. 26° C.).

TABLE 1
Composition of the AB Trace Medium used for the RDR test.

| | | Concentration |
|---|---|---|
| Component | Formula | (g/l) |
| Disodium phosphate | Na₂HPO₄ | 6.0 |
| Monopotassium phosphate | KH₂PO₄ | 3.0 |
| Sodium Chloride | NaCl | 3.0 |
| Ammonium sulfate | (NH₄)₂SO₄ | 2.0 |
| Magnesium chloride | MgCl₂ | 0.2 |
| Glucose | C₆O₁₂H₆ | 0.054 |
| Calcium chloride | CaCl₂ | 0.010 |
| Sodium sulfate | Na₂SO₄ | 0.011 |
| Ferric chloride | FeCl₃ | 0.00050 |

For each test, two RDRs were operated in parallel with one receiving test compound and the other serving as an untreated control. The RDRs were sterilized by autoclave, then filled with sterile medium and inoculated with P. aeruginosa strain PAO1. The reactors were then incubated at room temperature in batch mode (no medium flow) for a period of 24 hours, after which the flow was initiated for a further 24 hour incubation. Test compounds were dissolved in 10 ml ethanol to achieve a concentration of 1.8 mg/ml. After the 48 hours of biofilm development described above, the 10 ml of ethanol containing the test compounds were added to the reactor to achieve a final concentration of approximately 50, 100, or 200 μg/ml. Control reactors received 10 ml of ethanol. The reactors were then incubated for an additional 24 hours in batch (no flow) mode. After this incubation period, the six coupons were removed from each reactor and placed in 12-well polystyrene tissue culture plates with wells containing either 2 ml of a 100 μg/ml tobramycin solution or 2 ml of phosphate-buffered saline (PBS). These plates were incubated at room temperature for two hours. The coupons were then rinsed by three transfers to plates containing 2 ml of fresh PBS. For each two RDR reactors run in parallel, four sets of three coupons were obtained: one set with no test compound treatment and no tobramycin treatment, one set with no test compound treatment and tobramycin treatment, one set treated with a test compound treatment and no tobramycin treatment, and one set treated with a test compound treatment and tobramycin. After rinsing, one coupon of each set of three was stained with a LIVE/DEAD® BacLight™ Bacterial Viability Kit (Molecular Probes, Eugene Oreg.) and imaged using epifluorescent microscopy. The remaining two coupons were placed in 10 ml of PBS and sonicated for five minutes to remove and disperse biofilm cells. The resulting bacterial suspensions were then serially diluted in PBS and plated on tryptic soy agar plates for enumeration of culturable bacteria. The plates were incubated for 24 hours at 37° C. before colony forming units (CFU) were determined.

The treatments of the individual test compounds with and without tobramycin are listed in Table 2. The results are averages from experiments performed on three separate days for each test compound. The values reported are as log₁₀ CFU.

TABLE 2

| | Asiatic | Asiatic | Asiatic | Madecassic | Corosolic |
|---|---|---|---|---|---|
| | acid | acid | acid | acid | acid |
| Test | 50 μg | 100 μg/ml | 200 μg/ml | 100 μg/ml | 100 μg/ml |
| Compound | /ml | | | | |
| Concen- | | | | | |
| tration | | | | | |
| Tobramycin | 5.3 | 5.5 | 5.2 | 5.5 | 4.2 |
| and Test | | | | | |
| Compound | | | | | |
| Test | 7.7 | 7.7 | 7.5 | 7.5 | 7.3 |
| Compound | | | | | |
| Tobramycin | 5.8 | 6.5 | 6.1 | 6.7 | 6.5 |
| Control | 7.5 | 7.8 | 7.6 | 8.0 | 7.9 |

The results clearly demonstrate the abilities of asiatic acid, corosolic acid, and madecassic acid to increase the biofilm's susceptibility to tobramycin by modifying the biofilm. In combination with tobramycin these test compounds demonstrated an additional reduction of 67% to 99% CFU when compared to tobramycin alone. This translates into a reduction of approximately 1,000,000 to 4,500,000 cells of P. aeruginosa at 100 μg/ml.

As a comparison to multiple published clinical studies, these results with asiatic acid, corosolic acid, or madecassic acid in combination with tobramycin demonstrate that improved lung function (FEV or forced expiratory volume) and decreased average CFU (density) in sputum from patients with cystic fibrosis would be observed in a combination therapy involving these compounds (Ramsey, Bonnie W. et. al., “Intermittent administration of inhaled tobramycin in patients with cystic fibrosis”, New England J. Medicine 340(1):23-30, 1999; Saiman, L. “The use of macrolide antibiotics in patients with cystic fibrosis”, Curr Opin Pulm Med, 2004, 10:515:523; Pirzada, O. et al. “Improved lung function and body mass index associated with long-term use of Macrolide antibiotics.”, J. Cystic Fibrosis, 2003, 2, p. 69-71). Using the endpoints listed in these publications and used in cystic fibrosis clinical trials, this example demonstrates that a combined treatment of tobramycin and a compound of the invention would provide benefit to cystic fibrosis patients or other people suffering from chronic lung infections. The research results of this example also demonstrate that the compounds of the invention in combination with an antibiotic would remove biofilms from teeth, skin, tissues, catheters, medical devices, and other surfaces.

### Example 6

Effect of Asiatic acid on Biofilm Growth and Inhibition with Streptococcus mutans 25175 and Streptococcus sobrinus 6715.

Asiatic acid was tested against S. mutans 25175 and S. sobrinus 6715 at a concentration of 40 ug/ml using the method described in Example 1. The use of 1 mL polycarbonate tubes were used in place of 96 well polysterene microtiter plates.

Testing asiatic acid at 40 μg/mL against S. mutans 25175 and S. sobrinus 6715 showed greater than 75 % biofilm growth inhibition.

### Example 7

The Effects of Asiatic Acid, Corosolic Asid, and Ursolic Acid on the Binding to and Invasion of E. coli Clinical Strain UTI89 Against Bladder Epithelial Cells

The effect of test compounds on bacterial invasion of E. coli clinical strain UTI89 was studied as described in Elsinghorst, et al. 1994, Methods Enzymol, 236:405-420; and Martinez et al., 2000, EMBO J., 19:2803-2812. Epithelial bladder cells were grown in plates. Asiatic acid, corosolic acid, or ursolic acid were added at concentrations of 10 μg/ml, 20 μg/ml, or 40 μg/ml to bacteria and epithelial cells for approximately 5, 15, 30, or 60 minutes with approximately 10⁷ CFU of E. coli. Binding was assessed at time zero and invasion was assessed at approximately 5, 15, 30, or 60 minutes from completing the mixture of compound, bacteria, and epithelial cells. As a control ethanol was added to cells to a final concentration of 0.1%. The effect of bacterial viability and bacterial adherence during the infection period was evaluated according to the methods described in Martinez et al., 2000, EMBO J., 19:2803-2812. The test compounds did not affect the binding of E. coli to bladder epithelial cells. The test compounds reduced the invasion of E. coli into bladder epithelial cells.

40 μg/ml of corosolic acid with bacteria and epithelial cells for 60, 15, and 5 minutes reduced invasion of E. coli into bladder epithelial cells by 90%, 70%, and 10%, respectively, as compared to the controls. These experiments were performed in triplicate. Furthermore and separately, 40 μg/ml and 20 μg/ml of corosolic acid with bacteria and epithelial cells for 60 minutes reduced invasion of E. coli into bladder epithelial cells by 90% (n=7) and 65% (n=4), respectively, as compared to the controls. These experiments demonstrate a dose and time dependent effect of corosolic acid interrupting the pathogenesis cycle of E. coli. 40 μg/ml of asiatic acid and ursolic acid with bacteria and epithelial cells for 60 minutes reduced invasion of E. coli into bladder epithelial cells by 87% (n=7) and 76% (n=4), respectively.

The present invention demonstrates that corosolic acid, asiatic acid, ursolic acid, and other compounds of the present invention reduce invasion of E. coli into bladder epithelial cells and therefore interrupt the pathogenesis of E. coli in bladder epithelial cells. The cycle of pathogenesis of E. coli in recurrent urinary tract infections involves repeated invasions allowing the bacteria to survive and persist in the host. The invasion of E. coli into the bladder epithelial cells enables them to resist the mammalian immune response, which allows the bacteria to re-invade deeper into host's tissues. The compounds interrupt a key point in the bacteria's life cycle.

### Example 8

The Effects of a cysB Deletion Mutant of E. coli Clinical Strain UTI89 on the Binding to and Invasion into Bladder Epithelial Cells

The method described in Example 6 was used to examine the binding and invasion of E. coli UTI89 cysB⁻ (described in Example 2) into bladder epithelial cells.

E. coli UTI89 cysB⁻ exhibited about 93% reduction of invasion into bladder epithelial cells as compared to wild type. The invasion of E. coli UTI89 cysB⁻ into bladder epithelial cells was slightly restored by plasmid complementation of cysB demonstrating only a 70% reduction of invasion as compared to wild type.

These experiments demonstrate that the cysB gene plays a vital role in the pathogenesis of clinical strains of E. coli. The compounds' modulation of a cysB gene interrupt the pathogenesis cycle of E. coli, thereby providing an effective means to treat chronic infections that involve biofilms.

### Example 9

Bladder Concentrations of Asiatic Acid and Madecassic Acid in Rats

Pharmacokinetic studies of asiatic acid and madecassic acid in rats were performed separately. Asiatic acid and madecassic acid were evaluated at 50 mg/kg (oral). Two animals were assigned to each group. Prior to dosing, a baseline blood sample was taken from each animal. At time zero, asiatic acid and madecassic acid, a single bolus dose in 50% Labrasol (Gattefosse) was given to each animal. Bladders were analyzed at 24 hours. Concentrations of both asiatic acid and madecassic acid in the bladder were approximately 30 μg/g at 24 hours. Asiatic acid and madecassic acid significantly reduced bacterial invasion within 15 minutes of administration.

These experiments demonstrate that asiatic acid and madecassic acid are in adequate concentrations in the bladders of mice to reduce invasion of bacteria and the formation of biofilms.

### Example 10

The Effects of Asiatic Acid, Corosolic Asid, and Ursolic Acid on the Pathogenesis of E. coli clinical strain UTI89 in Mice

The experiment was performed using the procedures described in Justice, S. et al., Differentiation and development pathways of uropathogenic Escherichia coli in urinary tract pathogenesis.PNAS, 2004, 101(5), p. 1333-1338. Briefly, E. coli UTI89[pCOMGFP] was prepared after retrieval from frozen stocks by inoculating in LB medium statically for approximately 20 hours. Cells were harvested and suspended in 1 ml of PBS. Cells were diluted to achieve approximately a 10⁸ CFU or 10⁷ CFU input into C3H/HeN mice (2 mice per group).

Mice were deprived of water for approximately two hours. In experiment 1, all mice were anesthetized with 0.15 cc ketamine cocktail. In experiment 2, all mice were anesthetized with isofluorane. In experiment 1, urine was dispelled from the bladders and approximately 40 μg/ml of test compound or an appropriate amount of ethanol as control was introduced into the bladders via catheterization of the urethra using a tubing coated tuberculin syringe. 30 minutes was allowed to elapse. In experiment 2, bladders were not pre-incubated with test compounds. Bladders were then expelled and an inoculum of 10⁸ CFU (Experiment 1) or 10⁷ CFU (Experiment 2) of E. coli containing 40 μg/ml of test compound or equivalent amount of ethanol as controls were introduced into the bladders as indicated above.

In experiment 1 five hours elapsed and in experiment 2 six hours elapsed, and then mice were anesthetized and sacrificed. The bladders were removed, bisected, stretched, and fixed in 3% paraformaldehyde for 1 hour at room temperature. Bladders were then permeabilized in 0.01% Triton/PBS for 10 minutes and counter stained with TOPRO3 (Molecular Probes) for 10 minutes for visualization by confocal microscopy. Bladders were mounted on Prolong antifade (Molecular Probes).

In experiment 1, corosolic acid, asiatic acid, and ursolic acid demonstrated a 94%, 77%, and 70% reduction, respectively, in biofilm pods or intracellular bacterial communities (IBC) in the bladders of mice as compared to the controls by examination with confocal microscopy. In experiment 2, both corosolic acid and asiatic acid demonstrated approximately a 60% reduction in large biofilm pods or large IBC in the bladders of mice as compared to the controls by examination with confocal microscopy.

The results of these experiments demonstrate that the compounds of the present invention interrupt the pathogenesis of clinical strains of E. coli in mice. Therefore, the compounds of the present invention can have a significant impact on the treatment of chronic infections involving biofilms. Justice, S. et al. described that biofilm pods or IBC play an integral role in the recurrence of urinary tract infections (Justice, S. et al. Differentiation and development pathways of uropathogenic Escherichia coli in urinary tract pathogenesis. PNAS, 2004, 101(5), p. 1333-1338). The authors described that IBC or biofilms prevent the mammalian immune response from eradicating the bacterial population, thereby allowing the IBC and bacteria within the IBC to increase in number. Therefore by interrupting the pathogenesis of the bacteria, the compounds of the present invention can work in combination with the mammalian immune system and/or an antibiotic to reduce, prevent, treat, or eradicate the bacterial infections involving biofilms. This animal model is representative of chronic lung, ear, and sinus infections, acne, rosacea, and chronic wounds. It is also representative of the cycle of pathogenesis of other E. coli infections such as, but not limited to, pyelonephritis, prostatitis, meningitis, sepsis, and gastrointestinal infections.

### Example 11

The Effects of a cysB Deletion Mutant of E. coli Clinical Strain UTI89 on the Pathogenesis of E. coli in Mice

Experiments were conducted as described in Example 9. A 50:50 mix of E. coli UTI89 cysB⁻[pCOMRFP] and wild type E. coli UTI89[pCOMGFP] was prepared and inoculated into 2 mice. Wild type E. coli UTI89[pCOMGFP] alone was inoculated into 2 control mice. At 6 hours, bladders were prepared accordingly for examination by confocal microscopy.

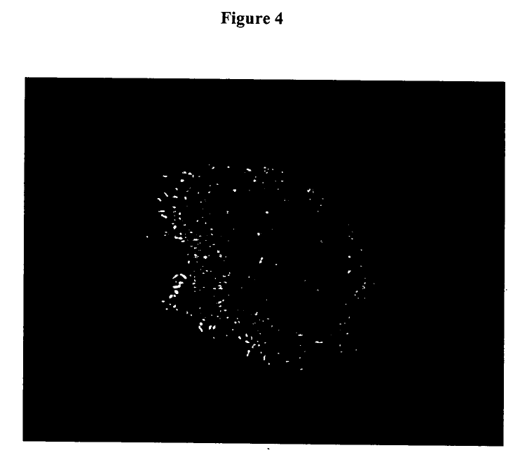

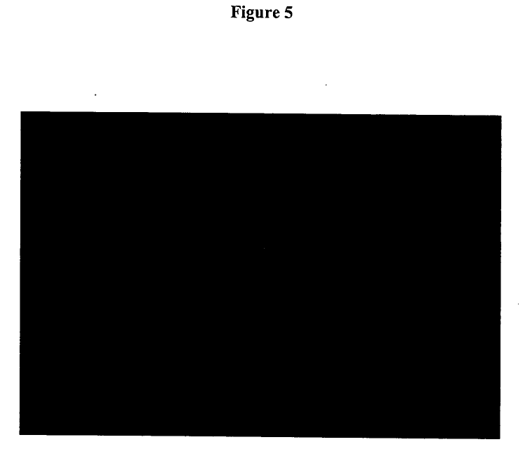

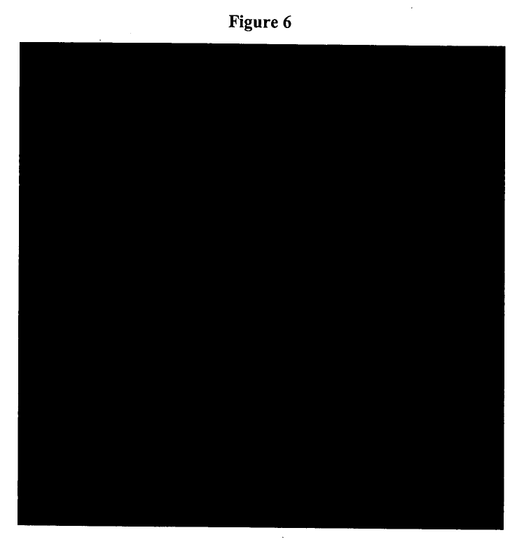

As can be seen in FIG. 4, the control bladders had typical IBC populations (or biofilms) similar to those published in Justice, S. et al. 2004. The bladders from the mice inoculated with the mutant/wild type mix showed populations of bacteria that exist in loose diffuse collections as shown in FIGS. 5 and 6. The collections of bacteria were markedly different from the control IBC.

Consistent with the teachings in Justice, S. et al. 2004, the loose collection of bacteria observed in FIGS. 5 and 6 would not be able to provide the bacteria with protection from leukocyte phagocytosis in the tissues of bladders; the bacteria no longer exist in dense, protective homogenous communities. Therefore, the cysB gene in E. coli enables the bacteria to form biofilms in the tissues of bladders. The cysB gene is genetically conserved amongst Gram-negative bacteria. Therefore, it is contemplated that modulation of this gene by the compounds of the present invention would also reduce the formation of biofilms in chronic infections caused by other bacteria besides E. coli.

### Example 12

A Topical Gel was Prepared Containing 2% of Madecassic Acid by Weight with Azithromycin for Use in Treating Acne, Rosacea, and Skin Infections

0.25 gram of madecassic acid was dissolved in 6.75 grams of ethanol. Then, 0.2 grams of azithromycin was dissolved in this solution. 0.25 grams of hydroxypropyl methylcellulose was added with gentle stirring until a homogenous solution was obtained. 4.8 grams of water was then added with gentle shaking.

This formulation was stored for thirty days at 2° C. to 8° C., room temperature (approximately 22° C.), and at 30° C. It remained homogenous for thirty days at each storage condition. A formulation without antibiotic could also be prepared using this same procedure.

### Example 13

Madecassic Acid, Pharmaceutical Formulation for Nebulization

Solutions were prepared comprising 2 mg/ml and 10 mg/ml of madecassic acid in ethanol/propylene glycol/water (85:10:5). These solutions were nebulized separately by a ProNeb Ultra nebulizer manufactured by PARI. The nebulized solutions were collected in a cold trap, processed appropriately, and detected by mass spectrometry. Madecassic acid was recovered from both formulations demonstrating that nebulization can be used to deliver this compound to patients with lung infections.

### Example 14

Madecassic Acid, 2% Toothpaste Formulation

Toothpaste preparations were prepared containing 2% madecassic acid with and without antibiotic and with and without polymer. In one embodiment, polymer, Gantrez S-97, was added to improve retention of madecassic acid and antibiotic on teeth.

All of the dry ingredients were mixed together. Glycerin was slowly added while mixing. An aliquot of water was added slowly and thoroughly mixed. Peppermint extract was added and then the rest of the water was added while mixing. Madecassic acid and antibiotic were then added until homogenous.

Formulation A

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Madecassic Acid | 2.0 |
| Peppermint extract | 1.0 |
| Sodium fluoride | 0.3 |
| Water | 31.7 |

Formulation B

| Ingredients | Parts By Weight |
|---|---|
| Sorbitol | 20.0 |
| Glycerin | 22.0 |
| Silica | 20 |
| Sodium lauryl sulfate | 2.0 |
| Xanthum gum | 1 |
| Madecassic Acid | 2.0 |
| Triclosan | 0.3 |
| Peppermint extract | 1.0 |
| Sodium fluoride | 0.3 |
| Gantrez S-97 | 2.5 |
| Water | 28.9 |

Formulations A and B were prepared and stored for thirty days at 2° C. to 8° C., room temperature (approximately 22° C.), and at 30° C.

What is claimed:

1. A method for reducing or preventing the invasion of a bacterium into a tissue comprising modulating the expression of a cysB gene in the bacterium.

2. The method of claim 1, wherein the modulation of the cysB gene comprises contacting the tissue with a composition comprising a compound selected from the group of ursolic acid or asiatic acid, or a pharmaceutically acceptable salt of such compound, or hydrate of such compound, or solvate of such compound, an N-oxide of such compound, or combination thereof.

3. The method of claim 2, wherein the compound is corosolic acid, 30-hydroxyursolic acid, 20-hydroxyursolic acid, 2-hydroxyoleanolic acid, and madecassic acid.

4. The method of claim 2, wherein the compound is pygenic acid (A, B, or C), euscaphic acid, and tormentic acid.

5. The method of claim 1, wherein the compound modulates the expression of cysD.

6. The method of claim 1, wherein the compound modulates the expression of cysI.

7. The method of claim 1, wherein the compound modulates the expression of cysJ.

8. The method of claim 1, wherein the compound modulates the expression of cysK.

9. The method of claim 1, wherein the compound modulates the expression of ybiK.

10. The method of claim 1, wherein the compound modulates the expression of b0829.

11. The method of claim 1, wherein the compound modulates the expression of b1729.

12. The method of claim 1, wherein the compound modulates the expression of yeeD.

13. The method of claim 1, wherein the compound modulates the expression of yeeE.

14. The method of claim 1, wherein the bacterium is a Gram-negative bacterium.

15. The method of claim 14, wherein the bacterium is Escherichia coli.

16. The method of claim 14, wherein the bacterium is Pseudomonas aeruginosa.

17. The method of claim 14, wherein the bacterium is Haemophilus influenzae.

18. The method of claim 1, wherein the tissue is a mammalian tissue.

19. The method of claim 18, wherein the mammalian tissue is a murine tissue.

20. The method of claim 18, wherein the mammalian tissue is a human tissue.

21. The method of claim 20, wherein the human tissue is a bladder.

22. The method of claim 20, wherein the human tissue is a kidney.

23. The method of claim 20, wherein the human tissue is a prostate.

24. The method of claim 1, wherein the tissue is a plant tissue.

25. An in vivo method for reducing or preventing the formation of a biofilm in a tissue comprising modulating expression of a cysB gene in a cell capable of biofilm formation.

26. The method of claim 25, wherein the modulation of the cysB gene comprises contacting the tissue with a composition comprising a compound selected from the group of ursolic acid, or asiatic acid, or a pharmaceutically acceptable salt of such compound, or hydrate of such compound, or solvate of such compound, an N-oxide of such compound, or combination thereof.

27. The method of claim 26, wherein the compound is corosolic acid, 30-hydroxyursolic acid, 20-hydroxyursolic acid, 2-hydroxyoleanolic acid, and madecassic acid.

28. The method of claim 26, wherein the compound is pygenic acid (A, B, or C), euscaphic acid, and tormentic acid.

29. The method of claim 25, wherein the compound modulates the expression of cysD.

30. The method of claim 25, wherein the compound modulates the expression of cysI.

31. The method of claim 25, wherein the compound modulates the expression of cysJ.

32. The method of claim 25, wherein the compound modulates the expression of cysK.

33. The method of claim 25, wherein the compound modulates the expression of ybiK.

34. The method of claim 25, wherein the compound modulates the expression of b0829.

35. The method of claim 25, wherein the compound modulates the expression of b1729.

36. The method of claim 25, wherein the compound modulates the expression of yeeD.

37. The method of claim 25, wherein the compound modulates the expression of yeeE.

38. The method of claim 25, wherein the cell is a Gram-negative bacterium.

39. The method of claim 38, wherein the cell is Escherichia coli.

40. The method of claim 38, wherein the cell is Pseudomonas aeruginosa.

41. The method of claim 38, wherein the cell is Haemophilus influenzae.

42. The method of claim 25, wherein the tissue is a mammalian tissue.

43. The method of claim 42, wherein the mammalian tissue is a murine tissue.

44. The method of claim 42, wherein the mammalian tissue is a human tissue.

45. The method of claim 44, wherein the human tissue is a bladder.

46. The method of claim 44, wherein the human tissue is a kidney.

47. The method of claim 44, wherein the human tissue is a prostate.

48. The method of claim 25, wherein the tissue is a plant tissue.

49. A method for controlling or preventing a chronic bacterial infection in a subject in need thereof comprising modulating the expression of a cysB gene in a bacterium that causes or contributes to the chronic bacterial infection.

50. The method of claim 49, wherein the modulation of the cysB gene comprises administering to a subject in need thereof with an effective amount of a composition comprising a compound selected from the group consisting of ursolic acid or asiatic acid, or a pharmaceutically acceptable salt of such compound, or hydrate of such compound, or solvate of such compound, an N-oxide of such compound, or combination thereof.

51. The method of claim 50, wherein the compound is corosolic acid, 30-hydroxyursolic acid, 20-hydroxyursolic acid, 2-hydroxyoleanolic acid, and madecassic acid.

52. The method of claim 50, wherein the compound is pygenic acid (A, B, or C), euscaphic acid, and tormentic acid.

53. The method of claim 49, wherein the compound modulates the expression of cysD.

54. The method of claim 49, wherein the compound modulates the expression of cysI.

55. The method of claim 49, wherein the compound modulates the expression of cysJ.

56. The method of claim 49, wherein the compound modulates the expression of cysK.

57. The method of claim 49, wherein the compound modulates the expression of ybiK.

58. The method of claim 49, wherein the compound modulates the expression of b0829.

59. The method of claim 49, wherein the compound modulates the expression of b1729.

60. The method of claim 49, wherein the compound modulates the expression of yeeD.

61. The method of claim 49, wherein the compound modulates the expression of yeeE.

62. The method of claim 49, wherein the chronic bacterial infection is selected from the group consisting of urinary tract infection, gastritis, lung infection, ear infection, cystitis, pyelonephritis, arterial damage, leprosy, tuberculosis, benign prostatic hyperplasia, prostatitis, osteomyelitis, bloodstream infection, cirrhosis, skin infection, acne, rosacea, open wound infection, chronic wound infection, and sinus infection.

63. The method of claim 49, wherein the chronic bacterial infection results from an infection of a bacterium.

64. The method of claim 63, wherein the bacterium is a Gram-negative bacterium.

65. The method of claim 64, wherein the bacterium is selected from the group consisting of Escherichia coli, Pseudomonas aeruginosa, Haemophilus influenzae.

66. The method of claim 49, wherein the chronic bacterial infection causes an autoimmune disease in a mammal.

67. The method of claim 66, wherein the mammal is a human.
